SEQUENCE LISTING (10 sequences)

SEQ ID NO: 1 - protein, 12 aa, Artificial Sequence
  Synthetic peptide prepared in the lab
  MISC_FEATURE (6)..(6): The side chain amine of the lysine residue at position 6 is lipoylated
KQTARKSTGGWW

SEQ ID NO: 2 - protein, 10 aa, Artificial Sequence
  Synthetic peptide prepared in the lab
  MISC_FEATURE: The side chain amine of the lysine residue at position 4 is lipoylated
VKSKATNLWW

SEQ ID NO: 3 - protein, 12 aa, Artificial Sequence
  Synthetic peptide prepared in the lab
  MISC_FEATURE (6)..(6): The side chain amine of the lysine residue at position 6 is lipoylated
SDPIIKGSGTWW

SEQ ID NO: 4 - protein, 14 aa, Artificial Sequence
  Synthetic peptide prepared in the lab
  MISC_FEATURE (6)..(6): The side chain amine of the lysine residue at position 6 is lipoylated
SGASEKDIVHSGWW

SEQ ID NO: 5 - protein, 12 aa, Artificial Sequence
  Synthetic peptide prepared in the lab
  MISC_FEATURE (6)..(6): The side chain amine of the lysine residue at position 6 is lipoylated
EIETDKATIGWW

SEQ ID NO: 6 - protein, 12 aa, Artificial Sequence
  Synthetic peptide prepared in the lab
  MISC_FEATURE (6)..(6): The side chain amine of the lysine residue at position 6 is lipoylated
EIETDKAVVTWW

SEQ ID NO: 7 - protein, 12 aa, Artificial Sequence
  Synthetic peptide prepared in the lab
  MISC_FEATURE (6)..(6): The side chain amine of the lysine residue at position 6 is lipoylated
EIETDKTSVQWW

SEQ ID NO: 8 - protein, 11 aa, Artificial Sequence
  Synthetic peptide prepared in the lab
  MISC_FEATURE (5)..(5): The side chain amine of the lysine residue at position 5 is lipoylated
VQSDKASVTWW

SEQ ID NO: 9 - protein, 12 aa, Artificial Sequence
  Synthetic peptide prepared in the lab
  MISC_FEATURE (6)..(6): The side chain amine of the lysine residue at position 6 is lipoylated
EALPKKTGGPWW

SEQ ID NO: 10 - protein, 12 aa, Artificial Sequence
  Synthetic peptide prepared in the lab
  MISC_FEATURE (6)..(6): The side chain amine of the lysine residue at position 6 is lipoylated
ALESVKAASEWW

(19) United States
(12) Patent Application Publication
(10) Pub. No.: US 2022/0009915 A1
(43) Pub. Date: Jan. 13, 2022

(54) CHEMICAL PROBES AND METHODS OF USE THEREOF

(71) Applicant: City University of Hong Kong, Hong Kong (CN)

(72) Inventors: Hongyan Sun, Hong Kong (CN); Yusheng XIE, Hong Kong (CN); Rui WANG, Hong Kong (CN); Liang ZHANG, Hong Kong (CN)

(21) Appl. No.: 17/371,177

(22) Filed: Jul. 9, 2021

Related U.S. Application Data
(60) Provisional application No. 63/049,693, filed on Jul. 9, 2020.

(51) Int. Cl. C07D 413/12; C07D 413/14; C12Q 1/48; G01N 21/64; C12N 9/10

(52) U.S. Cl. CPC C07D 413/12; C07D 413/14; C12Q 1/48; G01N 2333/91057; C12N 9/1029; G01N 2021/6439; G01N 21/6428

(57) ABSTRACT

Provided herein are turn-on fluorescent chemical probes useful for monitoring and/or detecting lysine delipoylation activity in a sample including or suspected of including a delipoylation enzyme.

### CROSS REFERENCE TO RELATED APPLICATIONS

This application claims the benefit of priority of U.S. Provisional Application No. 63/049,693 filed on Jul. 9, 2020, which is hereby incorporated by reference in its entirety.

### TECHNICAL FIELD

The present disclosure generally relates to the area of chemical probes. More particularly, the present disclosure relates to turn-on fluorescent chemical probes useful for detecting and/or monitoring delipoylation activity in a sample, kits comprising the same, and methods of use thereof.

### BACKGROUND

Post-translational modifications (PTMs) of lysine residues are highly prevalent in living organisms and play important roles in regulating diverse biological processes such as gene transcription, DNA repair, chromatin structure modulation, and metabolism. Notable examples of lysine PTMs include methylation, acetylation, lipidation, ubiquitination, sumoylation, and others. Recently the discovery of numerous new lysine acylations, such as succinylation (Ksucc), crontonylation (Kcr), 2-hydroxyisobutyrylation (Khib), and β-hydroxybutyrylation (Kbhb), has further expanded the landscapes of lysine PTMs. Deciphering the regulatory mechanisms of these new lysine PTMs is important to further elucidate their biological functions. Research in this field has therefore seen tremendous development and attracted increasing attention in recent years.

Lysine lipoylation (Klip) is a highly conserved lysine PTM found in bacteria, viruses, and mammals. It plays a critical role in regulating cell metabolism. Klip is reported to occur on several essential metabolic multimeric complexes, including the branched-chain α-ketoacid dehydrogenase (BCKDH), the α-ketoglutarate dehydrogenase (KDH) complex, the pyruvate dehydrogenase (PDH) complex, and the glycine cleavage complex (GCV). Klip is required as an essential cofactor for maintaining the activity of these enzyme complexes. Malfunction of these metabolic complexes, on the other hand, can lead to numerous diseases. For instance, dysregulation of PDH activity has been linked to many diseases including metabolic disorders, cancer, Alzheimer's disease, and viral infection. Notwithstanding the important roles of lysine lipoylation in biology, its regulatory mechanisms, in particular the enzymes that catalyze the removal (“erasers”) of this PTM, are still poorly understood. In 2013, Denu, et al. screened the in vitro deacylation activity of sirtuins against histone peptides with various acyl modifications including lipoylation. However, there remains a lack of knowledge of lysine lipoylation regulation, such as detailed enzymatic activity and in vivo substrate specificity. Elucidating the regulatory mechanism of lysine lipoylation will help understand its roles in biology and various diseases.

In a recent seminal work, Cristea et al., discovered that Sirt4 could interact with the PDH complex using immunoenrichment methods. The study revealed that Sirt4 is the first mammalian enzyme that can modulate PDH activity through delipoylation in living cells. However, it was noted that the delipoylation activity of Sirt4 in vitro was rather weak, especially when compared with the deacetylation activity of sirtuins. This raises an intriguing question: whether there are other enzymes that can erase Klip more efficiently in the native cellular environment.

There thus exists a need for new chemical probes to aid in understanding the biological functions of lysine lipoylation and that address at least some of the aforementioned challenges.

### SUMMARY

Provided herein is a family of fluorogenic probes, exemplified by KTlip, which were designed to detect delipoylation activity in a continuous manner. KTlip enables quick and reliable determination whether a given protein possesses delipoylation activity.

In a first aspect, provided herein is a chemical probe of Formula I:

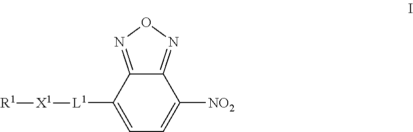

wherein X¹ is a moiety of Formula II:

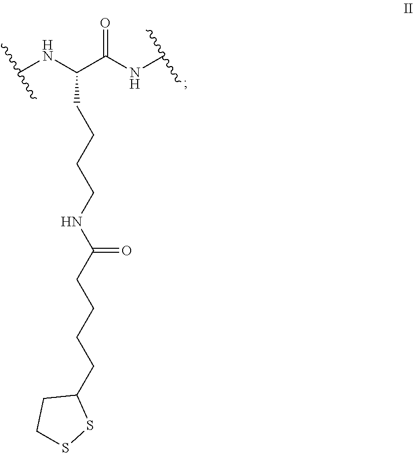 [SMILES: *NC(=O)[C@H](CCCCNC(=O)CCCCC1CCSS1)N*]

or

X¹ is a peptide sequence comprising an N-terminal amine and a C-terminal amine, wherein the peptide sequence is selected from the group consisting of branched-chain α-ketoacid dehydrogenase (BCKDH), α-ketoglutarate dehydrogenase (KDH), pyruvatedehydrogenase (PDH), glycine cleavage complex (GCV), and histone; and the peptide sequence comprises a lysine residue that is lipoylated represented by the moiety of Formula II;

R¹ is acetyl, tert-butyloxycarbonyl, or fluorenylmethoxycarbonyl; or R¹ is a moiety of Formula III:

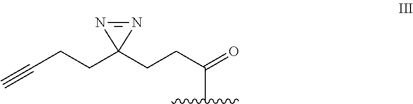

wherein R¹ is covalently bonded to the N-terminal amine of the peptide sequence; and L¹ is —(CH₂CH₂O)ₙ—, wherein n is 1, 2, or 3; and L¹ is covalently bonded to the C-terminal of the peptide sequence via an amide bond.

In certain embodiments, the peptide sequence is a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

In certain embodiments, n is 1 and R¹ is tert-butyloxycarbonyl.

In certain embodiments, the peptide sequence is a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10; n is 1; and R¹ is tert-butyloxycarbonyl.

In certain embodiments, the chemical probe has Formula IV:

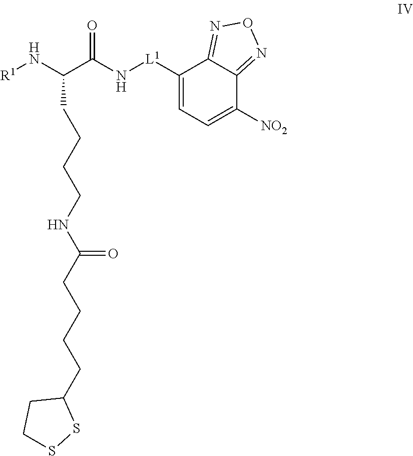

wherein R¹ is acetyl, tert-butyloxycarbonyl, or fluorenylmethoxycarbonyl; or R¹ is a moiety of Formula III:

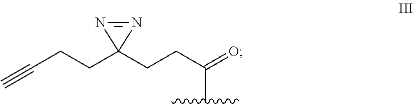 [SMILES: *C(=O)CCC1(CCC#C)N=N1]

and

L¹ is —(CH₂CH₂O)ₙ—, wherein n 1, 2, or 3.

In certain embodiments, n is 1.

In certain embodiments, R¹ is tert-butyloxycarbonyl.

In certain embodiments, the chemical probe has the Formula V:

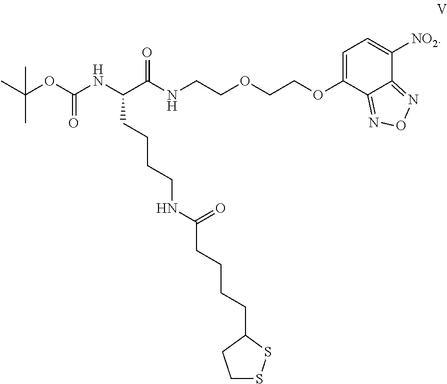 [SMILES: CC(C)(C)OC(=O)N[C@@H](CCCCNC(=O)CCCCC1CCSS1)C(=O)NCCOCCOc1ccc([N+](=O)[O-])c2nonc12]

In certain embodiments, the chemical probe has Formula VI:

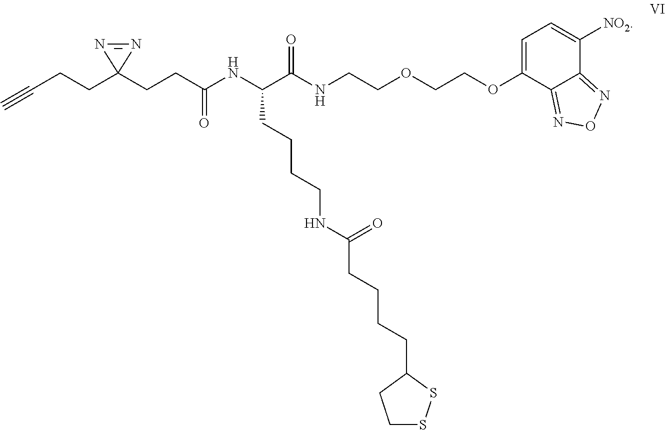 [SMILES: C#CCCC1(CCC(=O)N[C@@H](CCCCNC(=O)CCCCC2CCSS2)C(=O)NCCOCCOc2ccc([N+](=O)[O-])c3nonc23)N=N1]

In a second aspect, provided herein is a kit comprising a first container comprising a chemical probe as described herein; and a second container comprising nicotinamide adenine dinucleotide.

In a third aspect, provided herein is a method of detecting delipoylation activity in a sample comprising a delipoylation enzyme, the method comprising contacting the sample with a chemical probe described herein hereby forming a test sample; irradiating the test sample with light; and detecting the fluorescence of the test sample.

In certain embodiments, the delipoylation enzyme is a lysine delipoylation enzyme.

In certain embodiments, the delipoylation enzyme is a sirtuin (SIRT).

In certain embodiments, the delipoylation enzyme is SIRT2 or SIRT4.

In certain embodiments, the peptide sequence is a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10; n is 1; and R¹ is tert-butyloxycarbonyl.

In certain embodiments, the chemical probe has Formula V:

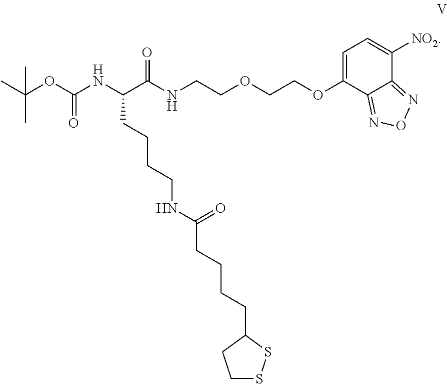 [SMILES: CC(C)(C)OC(=O)N[C@@H](CCCCNC(=O)CCCCC1CCSS1)C(=O)NCCOCCOc1ccc([N+](=O)[O-])c2nonc12]

In certain embodiments, the test sample is irradiated with light having an excitation wavelength of 480 nm.

In certain embodiments, the luminescence of the test sample is detected at an emission wavelength between 510-600 nm.

In certain embodiments, wherein the step of detecting the fluorescence of the test sample is done continuously.

In certain embodiments, the method comprises contacting a sample comprising a delipoylation enzyme selected from SIRT2 or SIRT4 with a chemical probe of Formula V:

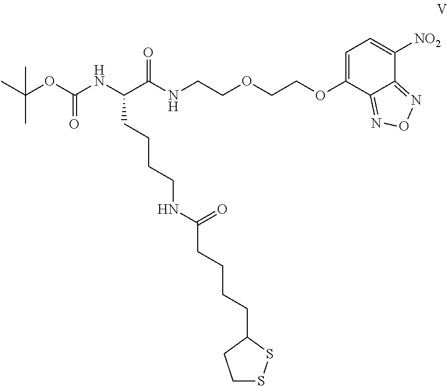 [SMILES: CC(C)(C)OC(=O)N[C@@H](CCCCNC(=O)CCCCC1CCSS1)C(=O)NCCOCCOc1ccc([N+](=O)[O-])c2nonc12]

thereby forming a test sample; irradiating the test sample with light having an excitation wavelength of 480 nm; and detecting the fluorescence of the test sample at an emission wavelength between 510-600 nm.

In alternative embodiments, provided herein is a lysine lipoylation probe comprising a recognition group and a reporting group, wherein a recognition group is at least one peptide from different lipoylated peptides. In one example, said different lipoylated peptides are selected from reported lipoylated proteins (PDH, KDH, BCKDH and GCV) and non-lipoylated proteins (e.g. histone). The lipoylated peptides may comprise a lysine residue, particularly a lysine residue with a lipoic acid functionalized thereon. The lipoylated peptides may also comprise an acetylated N-terminus. Additionally or optionally, the acetylated N-terminus may be replaced with a photo-crosslinker comprising a diazirine.

In another example, the reporting group is an O-nitrobenzoxadiazole (NBD) moiety. The O-NBD moiety may be converted to an N-NBD moiety and yield fluorescence when an enzyme hydrolyzes the lipoyl group on the lysine residue, of which the released form attacks the O-NBD moiety.

In certain embodiments, provided herein is a method of identifying a mammalian delipoylating enzyme probe comprising incubating mammalian delipoylating enzyme with one or more activity-based protein profiling reagents, at least one reagent comprising an amino acid peptide modified with lipoic acid, the lipoylated peptide and the reagent further comprising an O-NBD moiety.

In certain embodiments, provided herein is a method of competing a mammalian delipoylating enzyme probe comprising exposing mammalian delipoylating enzyme to a lysine lipoylation peptide comprising an amino acid peptide modified with lipoic acid, the lipoylated peptide.

In certain embodiments, provided herein is a method of negatively controlling a mammalian delipoylating enzyme probe comprising exposing mammalian delipoylating enzyme to a peptide comprising an amino acid peptide and an O-NBD moiety.

In certain embodiments, provided herein is a method of synthesizing a lysine lipoylation probe, comprising a standard Fmoc-based solid-phase chemistry on a peptide synthesizer.

The chemical probes described herein offer an efficient tool to evaluate the activity of lysine delipoylation of enzymes. The probe can quickly and reliably examine whether a given protein possesses delipoylation activity with simple procedure. In addition, it can be used to evaluate the potential of small compounds as inhibitors of delipoylation enzymes.

### BRIEF DESCRIPTION OF THE DRAWINGS

The above and other objects and features of the present disclosure will become apparent from the following description of the disclosure, when taken in conjunction with the accompanying drawings.

FIG. 1 depicts (A) Schematic diagram illustrating PDH catalyzes the conversion of pyruvate to acetyl-CoA, which is linked to the TCA cycle. We hypothesize that PDH activity might be inhibited by Sirt2 through delipoylation. (B) Relative PDH activity comparison between HeLa-S3 cells overexpressing Sirt2 and wild-type cells. PDH activity was measured by a commercial colorimetric assay. (C) Western blot analysis of endogenous lipoylated DLAT of PDH in cells overexpressing Sirt2 versus wild-type cells. DLAT is used as a loading control.

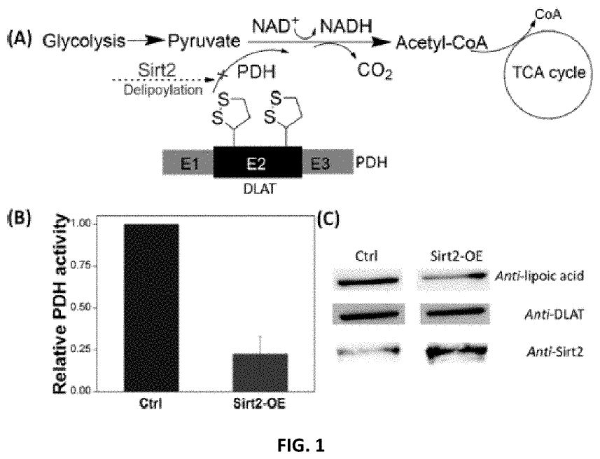

FIG. 2 depicts (A) Potential biological role of Sirt2 to erase lipoyl modification. (B) delipoylation on DLAT are linked to citric acid cycle and ATP synthesis.

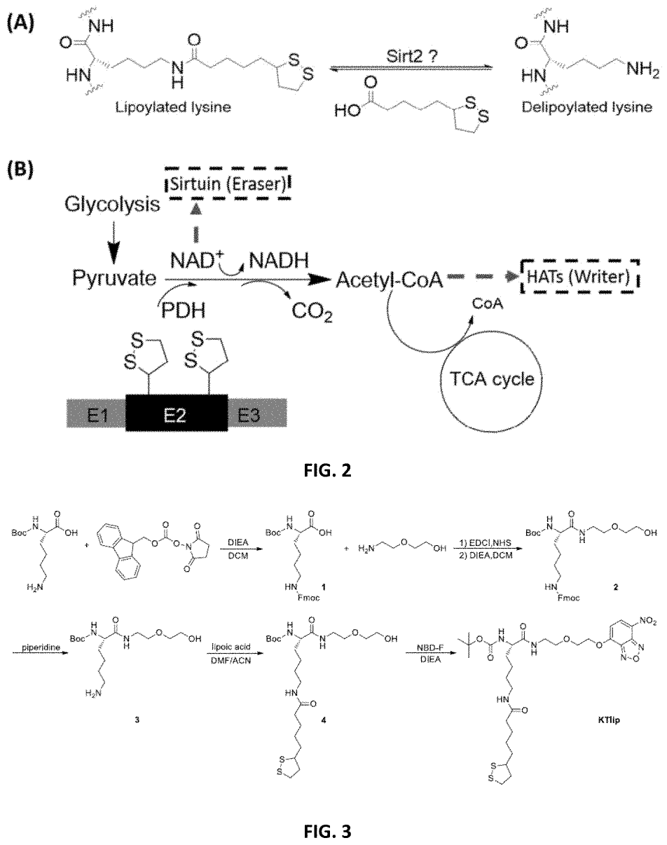

FIG. 3 depicts an exemplary synthesis of KTlip.

FIG. 4 depicts (A) HPLC analysis of the enzymatic reaction of KTlip (40 μM) with Sirt2 (80 ng/μl). The reaction was monitored at specified time at 254 nm. The retention time of the peaks was marked with asterisk 1 and 2 respectively (peak 1: 28.0 min, peak 2: 21.9 min). (B) ESI mass spectrum of the peak at 21.9 min in HPLC analysis. The mass peak corresponds to the tandem delipoylated/exchanged product. (C) Absorption spectra of KTlip (20 μM) before and after enzymatic reaction. Enzymatic reaction condition: Sirt2 (40 ng/μl), 200 μM NAD+ in 20 mM HEPES buffer (pH 8.0) at 37° C. for 2 h 30 mins.

FIG. 5 depicts (A) Lineweaver-Burk analysis for delipoylation of KTlip by Sirt2 using fluorescence assay method. (B) Lineweaver-Burk analysis for delipoylation of KTlip by Sirt2 using HPLC assay method.

FIG. 6 depicts HPLC analysis of the enzymatic reaction of KTlip (40 μM) with HDAC8 (80 ng/μl) in the reaction buffer (20 mM HEPES buffer at pH 8.0, 150 mM NaCl, 1 mM MgCl2 and 2.7 mM KCl). After 2 hours reaction, there was no delipoylated product observed.

FIG. 7 depicts (A) Comparison of relative PDH activity between Sirt2-knockdown and wild-type Hela cells. PDH activity was measured by a commercial colorimetric assay. (B) Western blot analysis of endogenous lipoylated DLAT of PDH in Sirt2-knockdown and wild-type Hela cells. DLAT is used as loading control.

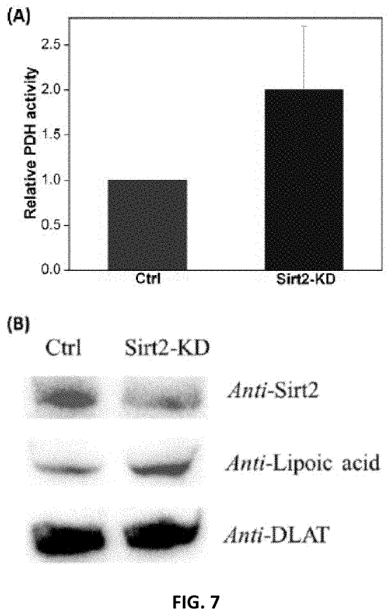

### DETAILED DESCRIPTION

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

It should be understood that the order of steps or order for performing certain actions is immaterial so long as the present teachings remain operable. Moreover, two or more steps or actions may be conducted simultaneously.

The use of the singular herein includes the plural (and vice versa) unless specifically stated otherwise.

The present disclosure provides chemical probes useful for detecting and/or monitoring delipoylation activity. In certain embodiments, the chemical probe has the Formula I:

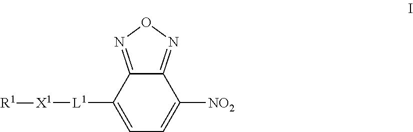

wherein X¹ is a moiety of Formula II:

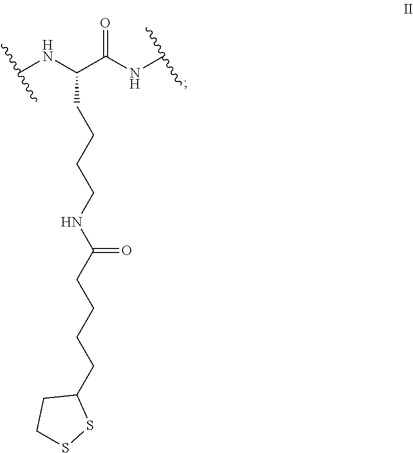 [SMILES: *NC(=O)[C@H](CCCCNC(=O)CCCCC1CCSS1)N*]

or

X¹ is a peptide sequence comprising an N-terminal amine and a C-terminal amine, wherein the peptide sequence is a polypeptide selected from the group consisting of branched-chain α-ketoacid dehydrogenase (BCKDH), α-ketoglutarate dehydrogenase (KDH), pyruvatedehydrogenase (PDH), glycine cleavage complex (GCV), and histone; and the peptide sequence comprises a lysine residue that is lipoylated represented by the moiety of Formula II;

R¹ is acetyl, tert-butyloxycarbonyl, or fluorenylmethoxycarbonyl; or R¹ is a moiety of Formula III:

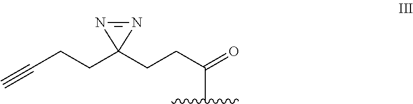

wherein R¹ is covalently bonded to the N-terminal amine of the peptide sequence; and L is —(CH₂CH₂O)ₘ—, with n is 1, 2, or 3, wherein L¹ is covalently bonded to the C-terminal of the peptide sequence via an amide bond.

In instances in which R¹ is moiety of Formula III, the chemical probe can be used to both monitor/detect delipoylation activity, but also label and identify proteins involved in delipoylating the chemical probe of Formula I.

The peptide sequence may comprise a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. In certain embodiments, the peptide sequence consists of a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

In certain embodiments, X¹ is a moiety of Formula II and the chemical probe can be represented by the chemical probe of Formula IV:

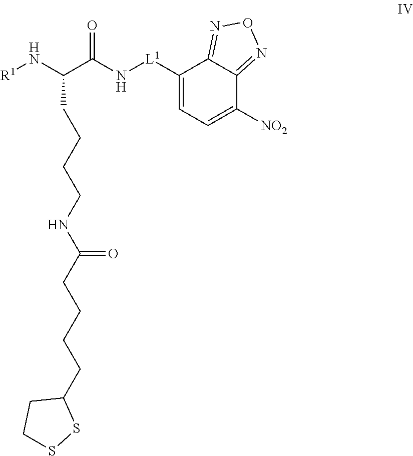

wherein R¹ is acetyl, tert-butyloxycarbonyl, or fluorenylmethoxycarbonyl; or R¹ is a moiety of Formula III:

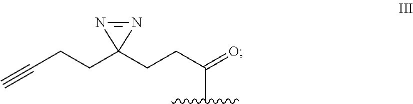 [SMILES: *C(=O)CCC1(CCC#C)N=N1]

and

L¹ is —(CH₂CH₂O)ₙ—, with n 1, 2, or 3.

In certain embodiments, the chemical probe has the Formula V (KTlip):

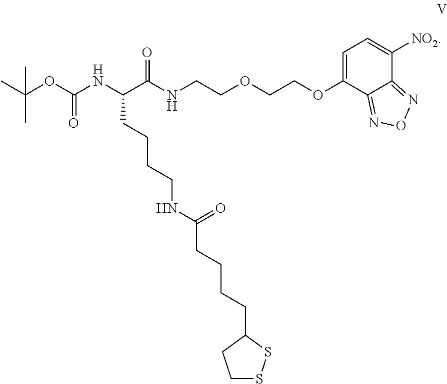 [SMILES: CC(C)(C)OC(=O)N[C@@H](CCCCNC(=O)CCCCC1CCSS1)C(=O)NCCOCCOc1ccc([N+](=O)[O-])c2nonc12]

In an exemplary example, the lysine lipoylation probe is KTlip. KTlip consists of a recognition group, lysine lipoylation and an O-NBD moiety. When enzymes hydrolyze the lipoyl group, the released amine attacks the O-NBD moiety, yielding an N-NBD moiety and turn on the fluorescence of KTlip. With the use of KTlip, the inventors have identified that Sirt2 (Sirtuin 2) is a novel lysine-delipoylation enzyme. Compared with the delipoylation activity of Sirt4, the only known mammalian lysine delipoylating enzyme, the present invention revealed that Sirt2 displays a more robust activity in removing lysine lipoylation in vitro.

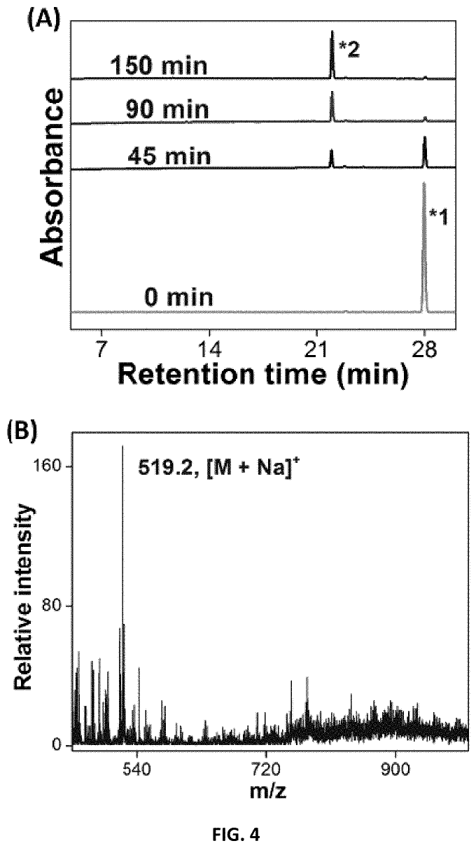

The chemical probes described herein can be readily prepared using well known synthetic methodology. In certain embodiments, the chemical probes are synthesized using solution phase chemistry. In alternative embodiments, the chemical probes are prepared using solid supported peptide synthesis methods. The synthesis of the chemical probes described herein are well within the skill of a person of ordinary skill in the art. An exemplary synthesis of chemical probe V is shown in FIG. 3.

Also provided herein is a kit comprising a first container comprising a chemical probe described herein; and optionally a second container comprising a co-factor, such as NAD+. The kit may optionally comprise instructions for carrying out the methods described herein.

The present disclosure also provides a method of detecting delipoylation activity in a sample comprising a delipoylation enzyme, the method comprising contacting the sample with a chemical probe described herein thereby forming a test sample; irradiating the test sample with light; and detecting the fluorescence of the test sample.

The sample can comprise a delipoylation enzyme or be suspected of comprising a delipoylation enzyme. The sample can be derived from a biological origin, such as a cell, tissue, cell culture, or the like.

Upon contacting the sample with the chemical probe described herein, at least a portion of the chemical probe can be delipoylated by the delipoylation enzyme thereby forming a delipoylated intermediate. The free lysine side chain of the delipoylated intermediate can take part in an intra-molecular or inter-molecular nucleophilic aromatic substitution reaction with the NBD thereby forming a compound of Formula VII:

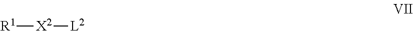

wherein X² is a moiety of Formula VIII:

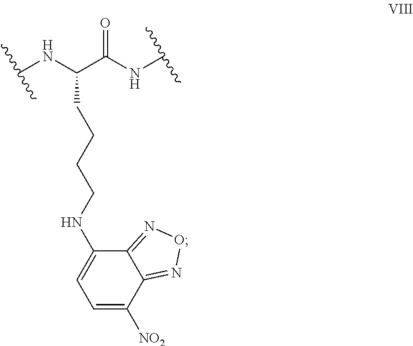 [SMILES: *NC(=O)[C@H](CCCCNc1ccc([N+](=O)[O-])c2nonc12)N*]

or

X² is a peptide sequence comprising an N-terminal amine and a C-terminal amine, wherein the peptide sequence is a polypeptide selected from the group consisting of branched-chain α-ketoacid dehydrogenase (BCKDH), α-ketoglutarate dehydrogenase (KDH), pyruvatedehydrogenase (PDH), glycine cleavage complex (GCV), and histone; and the peptide sequence comprises a lysine residue that is arylated represented by the moiety of Formula VIII;

R¹ is acetyl, tert-butyloxycarbonyl, or fluorenylmethoxycarbonyl; or R¹ is a moiety of Formula III:

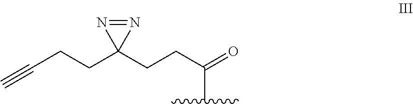

wherein R¹ is covalently bonded to the N-terminal amine of the peptide sequence; and L² is —(CH₂CH₂O)ₙH, wherein n is 1, 2, or 3 and L¹ is covalently bonded to the C-terminal of the peptide sequence via an amide bond.

Formation of the compound of Formula VII would cause the irradiated test sample to luminesce. The test sample can be irradiated at an excitation wavelength of the compound of Formula VII, e.g., 480 nm, causing the compound of Formula VII to luminesce, e.g., at an emission wavelength of 510-600 nm.

The step of detecting the fluorescence of the test sample can be accomplished by visual inspection and/or using a spectrometer. Any conventional spectrometer capable of measure absorbance of the test sample, which can fall between about 450 to 700 nm can be used. In certain embodiments, the spectrometer is a visible light spectrometer that is capable of measuring absorbance of the test sample between 450 to 700; 500 to 650 nm; or 510 to 600 nm.

The methods described herein can be used to detect and/or continuously monitor delipoylation activity of a delipoylation enzyme.

The delipoylation enzyme can be any enzyme capable of directly or indirectly delipoylating a chemical probe described herein. In certain embodiments, the delipoylation enzyme is SIRT, such as SIRT2 or SIRT4.

Delipoylation Study with Probe KTlip.

After confirming the interactions of Sirt2 and lipoylated peptide in cellular context, it was determined whether Sirt2 is capable of removing lipoyl modification. To this end, fluorescent probes were designed to detect delipoylation activity. Compared with mass spectrometry, radioisotopes, specific antibodies, and HPLC, fluorescent probes possess prominent advantages in detecting enzyme activity, such as high sensitivity and simple procedure. Until now, no single-step fluorescent probe has been developed to report delipoylation activity. In fact, it is difficult to design single-step fluorescent probes for detecting deacylation activity, because the aliphatic amide structure in the Kacyl group does not allow conjugation to a fluorophore.

A family of fluorogenic probes, exemplified by KTlip, for profiling delipoylation activity in vitro was designed. The probe includes a recognition group, Klip, and an 0-NBD moiety. It was hypothesized that when enzymes hydrolyze the lipoyl group, the released amine will attack the O-NBD (which can occur intra-molecularly or inter-molecularly), yielding NNBD, and turn on the fluorescence. Such a probe can report the delipoylation activity of enzymes continuously and reliably.

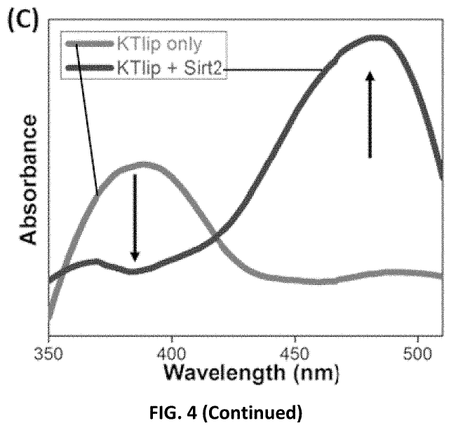

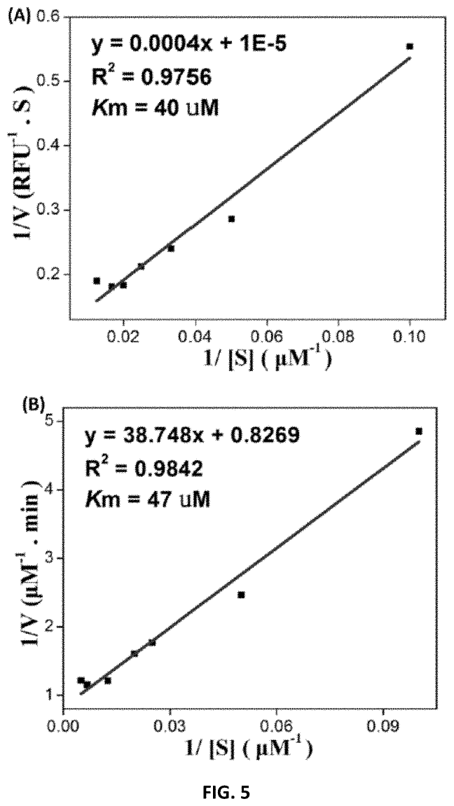

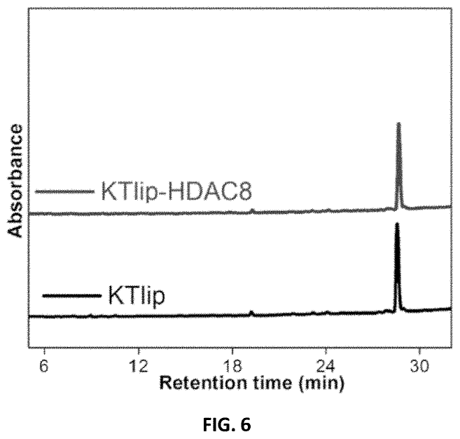

The probe KTlip following was first synthesized. The capacity of HDACs to recognize and remove the lipoyl group of KTlip was then examined by a fluorimeter assay. Briefly, KTlip was incubated with various HDACs at 37° C. in HEPES buffer (pH 8.0). The fluorescence of the enzymatic reactions was then measured accordingly. Sirt2 showed the strongest fluorescence increment, with 60-fold fluorescence increase. Sirt1 showed a much lower fluorescence signal, whereas Sirt3, Sirt5, Sirt6, and HDAC8 did not show a noticeable fluorescence increase. The control group without cofactor NAD+ displayed negligible fluorescence, indicating the reaction occurred through enzymatic catalysis. Further HPLC and MS analysis confirmed that the molecular weight of the newly generated peak corresponded to the expected tandem delipoylated/exchanged product (FIG. 4A,B). It was noted that no delipoylated product was observed under the enzymatic reaction conditions for HDAC8 (FIG. 6). After enzymatic reaction with Sirt2, a shift of peak absorption from 380 nm to 480 nm was clearly observed. (FIG. 4C). Through detailed kinetic study, the first-order rate constant of the reaction was determined to be 0.013 min−1. The Km value of KTlip obtained from the fluorescent method matched well with that from the traditional HPLC method (FIG. 5), underscoring that probe KTlip can serve as a useful tool for detecting enzymatic delipoylation activity. These results revealed that Sirt2 displays robust activity to remove the lipoyl group in vitro.

In conclusion, a panel of chemical probes were designed to investigate the regulatory mechanism of lysine lipoylation. KTlip is the first single-step fluorescent probe developed for rapid profiling of delipoylation activity. The enzymology data obtained from both KTlip and lipoylated peptides demonstrated the robust delipoylation ability of Sirt2 in vitro. It is noteworthy that the delipoylation activity of Sirt2 is far superior to that of Sirt4, the only identified mammalian delipoylating enzyme.

Through the chemical probes described herein, it the novel function of Sirt2 to remove the lipoyl group with high catalytic efficiency was shown. Furthermore, it was also demonstrated that Sirt2 could effectively catalyze DLAT delipoylation and downregulate PDH activity in cells. It is noted that a recent report showed that Sirt3 could enhance PDH activity through deacetylating the E1 subunit. This suggests that sirtuins might play a complex role in the dynamic regulation of PDH activity through different deacylation mechanisms. With the probes developed in this study, we envision that they will provide useful tools to further advance our understanding of lipoylation and other acylation in biology and diseases.

General Information.

Sirtuins, including Sirt1, Sirt2, Sirt3, Sirt5, and Sirt6, were recombinantly expressed and purified according to previous reports. Pyruvate dehydrogenase E2 (DLAT) (NM_001931) human recombinant protein was from ORIGENE. Streptavidin magnetic beads were purchased from New England Biolabs. In-gel fluorescence scanning experiments were performed with a FLA-9000 Fujifilm scanner. Antibody of Sirt2 (D4S6J) was from Cell Signaling. Antibodies of DLAT (ab172617), lipoic acid (ab58724), HDAC8 (ab187139), BRMS1L (ab107171), and Hsp60 (ab128567) were from Abcam. IRDye 680RD donkey anti-rabbit IgG (secondary antibody) was purchased from LI-COR Biosciences. Immobilon-FL poly(vinylidene difluoride) membrane for Western blotting was purchased from Merck Millipore. Western blotting was carried out with a C600 Azure biosystem. Sirt2 siRNA (AM16708) was from ThermoFisher Scientific. The plasmid pCMV4a-SIRT2-Flag was purchased from Addgene (plasmid #102623). The sequencing grade modified trypsin was purchased from Promega.

Absorption and Fluorescence Study of Probe KTlip.

The probe KTlip was incubated with sirtuin and NAD+ at 37° C. in 20 mM HEPES buffer (pH 8.0) containing 150 mM NaCl, 1 mM MgCl₂, and 2.7 mM KCl. The enzymatic reaction volume was 50 μL. When the enzymatic reaction was complete, the reaction was applied for absorption and fluorescence measurement. The parameter set for absorbance measurement was as follows: UV-visible light, collection region: 300-550 nm. The parameter set for fluorescence measurements was as follows: λex=480 nm, slit width: 5 nm, collection region: 510-600 nm.

Determination of the First-Order Rate Constant k.

It was calculated by fitting the fluorescence data to the following equation:

Fluorescence intensity=1−exp(−kt)

Enzymatic Reaction with Lipoylated Peptides.

The lipoylated peptides KAlip-1 to −11 were incubated with sirtuin and cofactor NAD+ at 37° C. in 20 mM HEPES buffer (pH 8.0) containing 150 mM NaCl, 1 mM MgCl₂, and 2.7 mM KCl. The reaction volume was set to 50 μL. At each specific reaction time point, the reaction mixtures were quenched by adding 250 μL of methanol. The reactions were vortexed and centrifuged. Supernatant was collected and then analyzed by reverse phase HPLC. The new peak generated was collected for ESI-MS or MALDI-TOF-MS analysis directly.

Kinetic Study with Lipoylated Peptides.

To determine the values of kcat and Km, purified Sirt2 with 400 μM NAD+ was incubated with different concentrations of lipoylated peptide (0-120 μM) in 20 mM HEPES buffer (pH 8.0) containing 150 mM NaCl, 1 mM MgCl2, and 2.7 mM KCl at 37° C. for 10 min (KAlip-1 and KAlip-10) or 5 min (KAlip-5 and KAlip-8). The reactions were quenched by adding 250 μL of methanol and then applied for HPLC analysis with a linear gradient of 5% to 85% B (acetonitrile) for 30 min. The generated delipoylated product was quantified based on the peak area monitored at 280 nm. The Km and kcat values were calculated by curve-fitting Vinitial/[E] versus [S]. The experiments were conducted in duplicate.

PDH Activity Assay.

To overexpress Sirt2 in cells, pCMV4aSirt2-Flag vector was transfected into HeLa-S3 cells using Lipofectamine 2000 (Invitrogen). The activity of PDH was assessed by measuring absorbance at 450 nm using a microplate assay kit (pyruvate dehydrogenase enzyme activity microplate assay, Abcam, ab109902). A 1000 μg amount of cell protein extracts was used for PDH immunocapture in each well. The experiments were performed in duplicate. Mitochondria Isolation. The mitochondrial fraction was isolated according to the manufacturer's instructions using a mitochondria isolation kit (Thermo Fisher, cat. No. 89874). The experiments were performed in duplicate.

General Procedure.

Starting materials and solvents were purchased from commercial suppliers and used without further purification, unless indicated otherwise. The required anhydrous solvents were purchased from J&K company or produced with common procedures. The required anhydrous conditions were carried out under nitrogen atmosphere using oven-dried glassware. Thin layer chromatography (TLC) for monitoring reaction was performed with pre-coated silica plates (Merck 60 F254 nm, 250 μm thickness), and spots were visualized by UV, phosphomolybdic acid, ninhydrin, or KMnO4 stain. Flash column chromatography was carried out with silica gel (Merck 60 F254 nm, 70-200 mesh). ¹H-NMR, ¹³C-NMR and ¹⁹F were recorded on Bruker 300 MHz/400 MHz NMR spectrometers. The spectra were referenced against the NMR solvent peaks (CD₃OD=3.31 ppm, CDCl3=7.26 ppm, CD3CN=1.94 ppm) and reported as follows: 1H: br (broad singlet), s (singlet), d (doublet), t (triplet), q (quartet), m (multiplet), dd (doublet of doublets). Mass spectra were obtained on a PC Sciex API 150 EX ESI-mass spectrometer or an Applied Biosystems 4800 Plus MALDI TOF/TOF analyzer.

pH value was measured with a HANNA HI 2211 pH/ORP meter. Fluorescence measurement was performed with a FluoroMax-4 fluorescence photometer. Absorption measurement was recorded with a UV-VS shimadzu 1700. Analytical high-performance liquid chromatography (HPLC) was carried out on a Waters 1525 Binary HPLC Pump and Waters 2489 UV/Visible Detector with a reverse phase Phenomenex Luna® Omega 5 μm Polar C18 100 Å 250×4.6 mm column at a flow rate of 1 mL/min. Acetonitrile and water were used as eluents. In-gel fluorescence scanning of the SDS-PAGE gels was carried out with a FLA-9000 Fujifilm system. Western blotting was carried out with a C600 Azure biosystem.

Determination of Michaelis Constant Km by HPLC and Fluorescence Method with Probe KTlip.

To determine Km with fluorescence method: a set of reactions with various concentrations of KTlip (0.1-100 μM) were incubated with recombinant Sirt2. The fluorescence was measured every 6 minutes (0-50 minutes). Fluorescence intensity was measured at 545 nm with excitation at 480 nm for each individual reaction. Finally, Km of Sirt2 was determined by plotting the reaction velocity against different substrate concentrations. For HPLC method, recombinant Sirt2 was incubated with different concentrations of KTlip (10, 20, 40, 50, 80, 150, 200 μM) and 500 μM NAD+ in 20 mM HEPES buffer (pH 8.0) containing 150 mM NaCl, 1 mM MgCl₂ and 2.7 mM KCl at 37° C. for 40 min. The reactions were quenched by adding 150 μL of methanol and then analyzed with reversed-phase HPLC. The substrate peaks were quantified with absorbance at 365 nm and converted to initial rates, which were then plotted against substrate concentration.

Cell Culture.

HEK-293, HeLa and HeLa-S3 cells were grown in Dulbecco's modified Eagle medium (DMEM) containing 10% heat-inactivated fetal bovine serum (FBS, Invitrogen), 100 μg/mL streptomycin, 100 units/mL penicillin, and sodium pyruvate (1 mM) (Thermo Scientific) at 37° C. in a humidified incubator with 5% CO₂.

Preparation of Cellular Lysates.

The cells were grown to 90% confluence and washed twice with cold phosphate-buffered saline (PBS). Lysis buffer (20 mM Tris-HCl, 500 mM NaCl, pH 7.5) was then added. The cells were harvested with a cell scraper and transferred to a 1.5 mL EP tube. They were subsequently lysed with sonication. Finally, the cellular lysates were centrifuged, and the supernatant was collected. Concentration of the proteins was determined by the bicinchoninic acid (BCA) assay.

Peptide Synthesis.

With exception of KAlip-2 to KAlip-11, which were purchased from commercial company Synpeptide in Shanghai, the other peptide derivatives were synthesized by standard Fmoc-based solid-phase chemistry on a CEM Liberty 1 peptide synthesizer. Rink-Amide resin (loading capacity: 0.6 mmol/g) was used as solid support. Coupling reactions were performed using 0.5 M 2-(1H-benzotriazole-1-yl)-1,1,3,3-tetramethyluroniumhexafluorophosphate (HBTU) and 1-Hydroxybenzotriazole hydrate (HOBt) in DMF as activator and 2 M N,N-diisopropylethylamine (DIPEA) as base in NMP. Each successive amino acid was used in 5-fold molar excess. 20% piperidine in DMF was used to remove the Fmoc group. Crude peptides were obtained by cleavage for 1.5 h using a cocktail containing TFA/triisopropylsilane (TIS)/H₂O (95:2.5:2.5). The peptides were further purified via preparative HPLC.

Synthesis of Compound 1

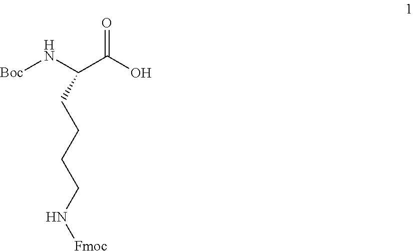

N,N-diisopropylethylamine (DIPEA, 517 g, 4 mmol) was added to the solution of Nα-(tert-Butoxycarbonyl)-lysine (493 mg, 2 mmol) and Fmoc N-hydroxysuccinimide ester (607 mg, 1.8 mmol) in anhydrous DCM, and the mixture was stirred overnight at r.t. After the reaction was complete, the solvent was removed under reduced pressure. The residue was purified by flash chromatography (EA/MeOH, 100/1) to afford the product 1 as a light yellow liquid (413 mg, 49% yield). ¹H NMR (DMSO, 400 MHz) (ppm): 12.41 (s, 1H), 7.89 (d, J=7.6 Hz, 2H), 7.68 (d, J=7.2 Hz, 2H), 7.41 (t, J=7.6 Hz, 2H), 7.33 (t, J=7.2 Hz, 2H), 7.28 (t, 1H), 7.04 (d, 1H), 4.3-4.28 (m, 2H), 4.22-4.19 (m, 1H), 3.82-3.79 (m, 1H), 2.98-2.93 (m, 2H), 1.66-1.37 (m, 15H). ¹³C-NMR (DMSO, 100 MHz) (ppm): 174.72, 156.54, 156.08, 144.42, 141.21, 128.06, 127.52, 125.61, 120.59, 78.41, 65.63, 60.22, 47.24, 31.17, 30.87, 29.43, 28.68, 23.38. ESI-MS calcd for [M−H]⁻ 467.23; Found 467.60.

Synthesis of Compound 2

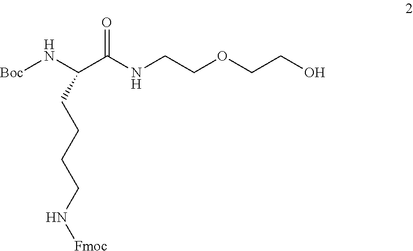

N-Hydroxysuccinimide (127 mg, 1.1 mmol) and N-(3-dimethylaminopropyl)-N′-ethylcarbodiimide hydrochloride (230 mg, 1.2 mmol) were added to a solution of compound 1 (413 mg, 0.88 mmol) in anhydrous DCM (2 mL). The mixture was stirred for 2 h at room temperature. N,N-diisopropylethylamine (336 mg, 2.6 mmol) and 2-(2-aminoethoxy)ethanol (116 mg, 1.1 mmol) were then added, and the mixture was stirred overnight. After the reaction was complete, the solvent was removed under reduced pressure. The residue was purified by flash chromatography (DCM/MeOH, 50/1) to afford product 2 as a colorless liquid (254 mg, 52% yield). ¹H NMR (CD₃OD, 400 MHz) (ppm): 7.77 (d, J=7.2 Hz, 2H), 7.62 (d, J=7.2 Hz, 2H), 7.37 (t, J=7.2 Hz, 2H), 7.29 (t, J=7.2 Hz, 2H), 4.32 (d, J=6.4 Hz, 2H), 4.16 (t, J=6.4 Hz, 1H), 3.99-3.96 (m, 1H), 3.63 (t, J=4.8 Hz, 2H), 3.49 (br, 4H), 3.38-3.35 (m, 2H), 3.11-3.07 (m, 2H), 1.71-1.41 (m, 15H). ¹³C-NMR (CD₃OD, 100 MHz) (ppm): 175.38, 158.60, 157.87, 145.45, 142.68, 128.85, 128.22, 126.24, 121.02, 80.67, 73.45, 70.56, 67.65, 62.27, 56.20, 41.45, 40.43, 33.22, 30.86, 30.55, 28.80, 24.14. ESI-MS calcd for [M⁺Na]⁺578.29; Found 578.6.

Synthesis of Compound 3

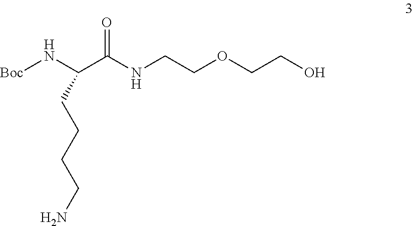 [SMILES: CC(C)(C)OC(=O)N[C@@H](CCCCN)C(=O)NCCOCCO]

1 mL of piperidine/DCM (1:1) was added to a solution of 2 (254 mg, 0.46 mmol). The reaction mixture was stirred at room temperature and monitored with TLC. After completion of the reaction, cold ethyl ether was added to precipitate product 3 as a sticky light yellow oil (114 mg, 75% yield). ¹H NMR (CD₃OD, 400 MHz) δ (ppm): 3.97 (br, 1H), 3.62 (t, J=4.8 Hz, 2H), 3.50-3.48 (m, 4H), 3.37-3.31 (m, 2H), 2.90-2.89 (m, 2H), 1.65-1.52 (m, 6H), 1.39 (s, 9H). ESI-MS calcd for [M⁺ H]⁺ 334.23; Found 334.6.

Synthesis of Compound 4

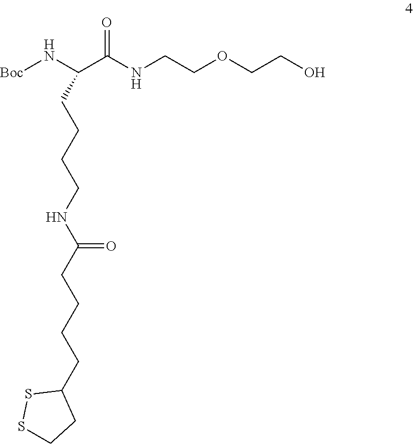 [SMILES: CC(C)(C)OC(=O)N[C@@H](CCCCNC(=O)CCCCC1CCSS1)C(=O)NCCOCCO]

1-Hydroxybenzotriazole hydrate (HOBt, 22 mg, 0.16 mmol) and N-(3-dimethylaminopropyl)-N′-ethylcarbodiimide hydrochloride (EDCI, 35 mg, 0.18 mmol) were added to a solution of lipoic acid (29 mg, 0.14 mmol) in anhydrous DMF (1 ml). The lipoic acid was activated for 1.5 h at room temperature. After that, a solution of N,N-diisopropylethylamine (DIPEA, 54 mg, 0.42 mmol) and compound 3 (54 mg, 0.16 mmol) in DMF (0.5 ml) was then added. And the mixtures were stirred overnight. After the reaction was complete, the solvent was removed under reduced pressure. The residue was purified by flash chromatography (DCM/MeOH, 70/1-20/1) to obtain the product 4 (63 mg, 86% yield). ¹H NMR (CD₃OD, 400 MHz) δ (ppm): 3.99-3.96 (m, 1H), 3.66 (t, J=4.4 Hz, 2H), 3.59-3.51 (m, 5H), 3.43-3.33 (m, 2H), 3.19-3.05 (m, 4H), 2.48-2.41 (m, 1H), 2.17 (t, J=7.2 Hz, 2H), 1.91-1.83 (m, 1H), 1.73-1.34 (m, 21H). ¹³C-NMR (CD₃OD, 100 MHz) (ppm): 175.93, 175.27, 157.78, 80.61, 73.43, 70.52, 62.24, 57.59, 56.11, 41.36, 40.40, 40.11, 39.42, 36.98, 35.78, 33.21, 30.11, 29.95, 28.82, 26.83, 24.32. ESI-MS calcd for [M⁺ H]⁺ 522.26; Found 522.6.

Synthesis of Compound KTlip

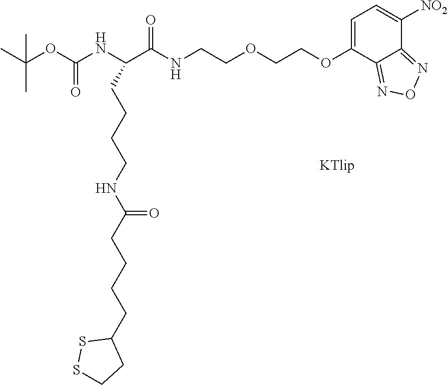 [SMILES: CC(C)(C)OC(=O)N[C@@H](CCCCNC(=O)CCCCC1CCSS1)C(=O)NCCOCCOc1ccc([N+](=O)[O-])c2nonc12]

N,N-diisopropylethylamine (67 mg, 0.52 mmol) and NBD-F (48 mg, 0.26 mmol) were added to a solution of 4 (63 mg, 0.12 mmol) in anhydrous DCM/DMF (3:1). The mixture was stirred overnight at r.t. After the reaction was complete, the solvent was removed under reduced pressure. The residue was purified by flash column chromatography (DCM/MeOH, 50/1-30/1), followed by preparative TLC, to obtain the product KTlip as a yellow solid (27 mg, 33% yield). ¹H NMR (CD₃OD, 300 MHz) δ (ppm): 8.64 (d, J=8.4 Hz, 1H), 6.99 (m, J=8.4 Hz, 1H), 4.58 (t, J=4.2 Hz, 2H), 3.99-3.93 (m, 3H), 3.66 (t, J=5.4 Hz, 2H), 3.59-3.34 (m, 3H), 3.19-3.03 (m, 4H), 2.49-2.39 (m, 1H), 2.16 (t, J=4.2 Hz, 2H), 1.92-1.81 (m, 1H), 1.72-1.32 (m, 21H). ¹³C-NMR (CD₃OD, 75 MHz) (ppm): 175.99, 175.44, 157.94, 156.02, 146.96, 145.61, 136.24, 131.07, 106.84, 80.64, 71.96, 70.91, 69.88, 57.65, 56.16, 41.41, 40.38, 40.13, 39.44, 37.01, 35.83, 33.16, 30.14, 30.00, 28.80, 26.87, 24.35. ESI-MS calcd for [M+H]+ 685.26; Found 685.80.

What is claimed is:

1. A chemical probe of Formula I: 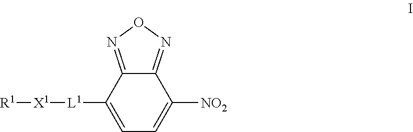
wherein X¹ is a moiety of Formula II: 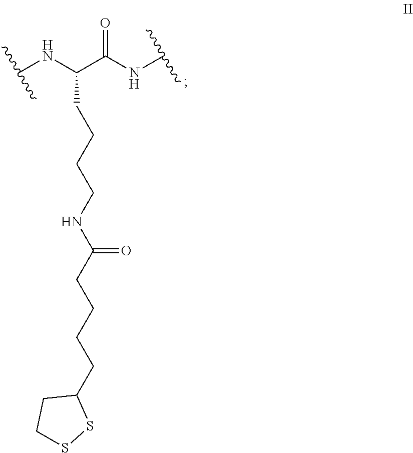 [SMILES: *NC(=O)[C@H](CCCCNC(=O)CCCCC1CCSS1)N*]
or
X¹ is a peptide sequence comprising an N-terminal amine and a C-terminal amine, wherein the peptide sequence is selected from the group consisting of branched-chain α-ketoacid dehydrogenase (BCKDH), α-ketoglutarate dehydrogenase (KDH), pyruvatedehydrogenase (PDH), glycine cleavage complex (GCV), and histone; and the peptide sequence comprises a lysine residue that is lipoylated represented by the moiety of Formula II;
R¹ is acetyl, tert-butyloxycarbonyl, or fluorenylmethoxycarbonyl; or R¹ is a moiety of Formula III: 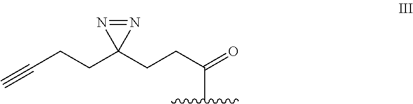
wherein R¹ is covalently bonded to the N-terminal amine of the peptide sequence; and L¹ is —(CH₂CH₂O)ₙ—, wherein n is 1, 2, or 3; and L¹ is covalently bonded to the C-terminal of the peptide sequence via an amide bond.

2. The chemical probe of claim 1, wherein the peptide sequence is a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

3. The chemical probe of claim 1, wherein n is 1 and R¹ is tert-butyloxycarbonyl.

4. The chemical probe of claim 1, wherein the peptide sequence is a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10; n is 1; and R¹ is tert-butyloxycarbonyl.

5. The chemical probe of claim 1, wherein the chemical probe has Formula IV: 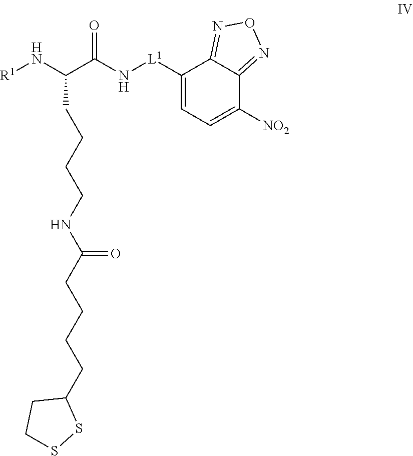
wherein
R¹ is acetyl, tert-butyloxycarbonyl, or fluorenylmethoxycarbonyl; or R¹ is a moiety of Formula III: 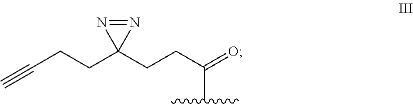 [SMILES: *C(=O)CCC1(CCC#C)N=N1]
and
L¹ is —(CH₂CH₂O)ₙ—, wherein n 1, 2, or 3.

6. The chemical probe of claim 5, wherein n is 1.

7. The chemical probe of claim 5, wherein R¹ is tert-butyloxycarbonyl.

8. The chemical probe of claim 1, wherein the chemical probe has the Formula V:

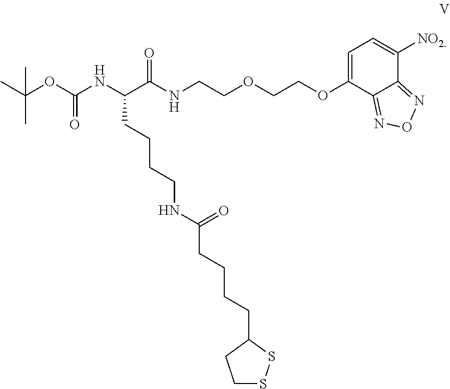 [SMILES: CC(C)(C)OC(=O)N[C@@H](CCCCNC(=O)CCCCC1CCSS1)C(=O)NCCOCCOc1ccc([N+](=O)[O-])c2nonc12]

9. The chemical probe of claim 1, wherein the chemical probe has Formula VI:

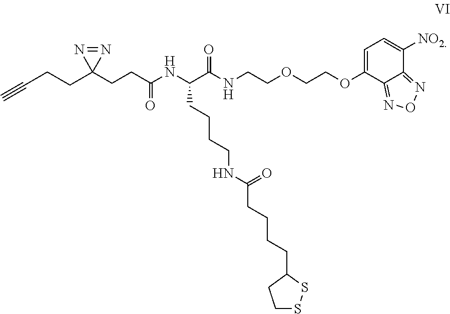 [SMILES: C#CCCC1(CCC(=O)N[C@@H](CCCCNC(=O)CCCCC2CCSS2)C(=O)NCCOCCOc2ccc([N+](=O)[O-])c3nonc23)N=N1]

10. A kit comprising a first container comprising a chemical probe of claim 1; and a second container comprising nicotinamide adenine dinucleotide.

11. A method of detecting delipoylation activity in a sample comprising a delipoylation enzyme, the method comprising contacting the sample with a chemical probe of claim 1 thereby forming a test sample; irradiating the test sample with light; and detecting the fluorescence of the test sample.

12. The method of claim 11, wherein the delipoylation enzyme is a lysine delipoylation enzyme.

13. The method of claim 11, wherein the delipoylation enzyme is a sirtuin (SIRT).

14. The method of claim 11, wherein the delipoylation enzyme is SIRT2 or SIRT4.

15. The method of claim 11, wherein the peptide sequence is a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10; n is 1; and R¹ is tert-butyloxycarbonyl.

16. The method of claim 11, wherein the chemical probe has Formula V:

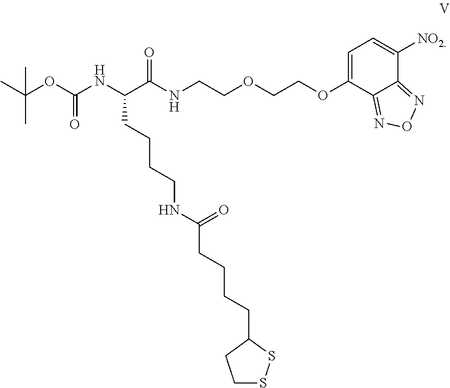 [SMILES: CC(C)(C)OC(=O)N[C@@H](CCCCNC(=O)CCCCC1CCSS1)C(=O)NCCOCCOc1ccc([N+](=O)[O-])c2nonc12]

17. The method of claim 11, wherein the test sample is irradiated with light having an excitation wavelength of 480 nm.

18. The method of claim 11, wherein the luminescence of the test sample is detected at an emission wavelength between 510-600 nm.

19. The method of claim 11, wherein the step of detecting the fluorescence of the test sample is done continuously.

20. The method of claim 11, wherein the method comprises contacting a sample comprising a delipoylation enzyme selected from SIRT2 or SIRT4 with a chemical probe of Formula V: 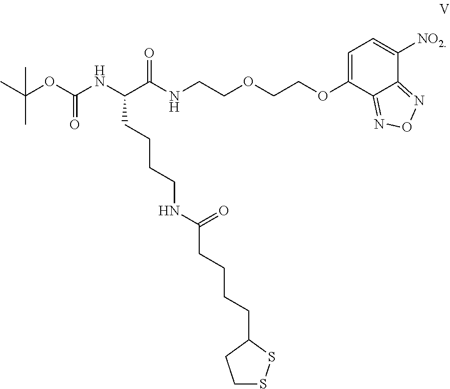 [SMILES: CC(C)(C)OC(=O)N[C@@H](CCCCNC(=O)CCCCC1CCSS1)C(=O)NCCOCCOc1ccc([N+](=O)[O-])c2nonc12]
thereby forming a test sample; irradiating the test sample with light having an excitation wavelength of 480 nm; and detecting the fluorescence of the test sample at an emission wavelength between 510-600 nm.
